(19) United States
(12) Patent Application Publication
(10) Pub. No.: US 2020/0254021 A1
(43) Pub. Date: Aug. 13, 2020

(54) Chimeric Antigen Receptor

(71) Applicant: UCL Business LTD, London (GB)

(72) Inventors: Martin Pulé, London (GB); Anne Kramer, London (GB); Evangelia K. Kokalaki, London (GB)

(21) Appl. No.: 16/796,370

(22) Filed: Feb. 20, 2020

Related U.S. Application Data
(63) Continuation of application No. 15/256,693, filed on Sep. 5, 2016, now abandoned.

(51) Int. Cl. A61K 35/17; C07K 14/705; C07K 14/725; C07K 16/28

(52) U.S. Cl. CPC A61K 35/17; C07K 14/70503; C07K 14/7051; C07K 16/2803; C07K 14/70578; C07K 2319/74; C07K 2317/622; C07K 2319/33; C07K 2319/03; C07K 2319/00; C07K 2319/02; C07K 14/70517

(57) ABSTRACT

The present invention provides a chimeric antigen receptor (CAR) comprising an antigen-binding domain with an affinity in the range of 50 nM to 500 nM, wherein said affinity comprises component kinetics such that the association rate constant (kₒₙ) is greater than or equal to 1×10⁵ M⁻¹ S⁻¹, and/or the dissociation rate constant (k_{off}) is greater than or equal to 0.01 s⁻¹.

### FIELD OF THE INVENTION

The present invention relates to a chimeric antigen receptor (CAR) comprising an antigen-binding domain with advantageous binding affinity. The invention also provides a method for selecting an antigen-binding domain for use in a chimeric antigen receptor, and a method for improving the ability of a CAR to mediate serial killing of target cells when expressed in a T cell. T cells expressing such a CAR are useful in the treatment of cancerous diseases such as B-cell leukemias and lymphomas.

### BACKGROUND TO THE INVENTION

Traditionally, antigen-specific T-cells have been generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem: transgenic expression of Chimeric Antigen Receptor (CAR) allows the generation of large numbers of T-cells specific to any surface antigen by ex vivo viral vector transduction of a bulk population of peripheral blood T-cells.

CARs are typically chimeric type I trans-membrane proteins which connect an extracellular antigen-recognizing domain (binder) to an intracellular signalling domain (endodomain) via a spacer and transmembrane domain. The binder is typically a single-chain variable fragment (scFv) derived from a monoclonal antibody (mAb). A spacer domain is necessary to isolate the binder from the membrane and to allow for suitable orientation, reach and segregation from phosphatases upon ligand engagement. A trans-membrane domain anchors the protein in the cell membrane and connects the spacer to the endodomain. The endodomain in a first generation CAR is commonly derived from the intracellular parts of either the γ chain of the FcεR1 or CD3ζ. Second and third generation CAR are generated from the addition of the endodomain from CD28 and/or OX40 or 41BB (which transmit proliferation and survival signals).

When challenged by tumour, CAR T-cells must effectively serially kill target cells, migrating rapidly between target cells and surviving unexhausted during this process. Optimized T-cell manufacturing processes which prevent exhaustion and differentiation of T-cells during production are important for achieving this aim. Despite optimization of CAR T-cell therapies for these factors, while CAR T-cells are effective in some patients, CAR T-cells often fail to function effectively. Thus there is still a need to improve the performance of CAR T-cells.

### SUMMARY OF ASPECTS OF THE INVENTION

The present inventors have surprisingly determined that a CAR derived from an antibody with a fast on-rate and a fast off-rate allows a CAR T-cell to better serially kill target cells. Therefore, CARs comprising antigen-binding domains with these properties are optimal for therapeutic purposes.

Thus, in a first aspect, the present invention provides a chimeric antigen receptor (CAR) comprising an antigen-binding domain with an affinity in the range of 50 nM to 500 nM, wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.01 s⁻¹.

The antigen-binding domain may have an affinity of about 100 nM.

The affinity may comprise component kinetics such that the association rate constant (kₒₙ) is from 1×10⁵ M⁻¹ s⁻¹ to 1×10⁷ M⁻¹ s⁻¹.

The affinity may comprise component kinetics such that the dissociation rate constant (k_{off}) is from 0.01 s⁻¹ to 0.5 s⁻¹.

The association rate constant (kₒₙ) may be about 6×10⁵ M⁻¹ s⁻¹, and/or the dissociation rate constant (k_{off}) may be about 0.07 s⁻¹.

The antigen-binding domain may be a scFV.

In another aspect the present invention provides a polynucleotide which encodes a CAR according to the present invention.

In a further aspect the present invention provides a vector which comprises a polynucleotide according to the present invention.

In another aspect the present invention provides a cell which comprises a CAR according to the present invention.

The cell may be a T cell or a natural killer (NK) cell.

In a further aspect the present invention provides a cell composition which comprises a plurality of cells according to the present invention.

In a further aspect the present invention relates to a method for making a cell according to the present invention, which comprises the step of transducing or transfecting a cell with a vector of the invention.

In a further aspect the present invention provides a method for making a cell composition according to the present invention which comprises the step of transducing or transfecting a sample of cells from a subject ex vivo with a vector of the invention.

In yet another aspect the present invention provides a pharmaceutical composition which comprises a cell or a cell composition according to the present invention, together with a pharmaceutically acceptable carrier, diluent or excipient.

In another embodiment the present invention relates to a method for selecting an antigen-binding domain for use in a chimeric antigen receptor (CAR), the method comprising:
(a) determining the affinity and affinity component kinetics of the antigen-binding domain; and
(b) selecting the antigen-binding domain for use in a CAR if it has an affinity in the range of 50 nM to 200 nM, wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.1 s⁻¹.

The method may comprise determining the affinity and affinity component kinetics of the antigen-binding domain of a plurality of antigen-binding domains.

The antigen-binding domain selected may be an antigen-binding domain as defined the first aspect of the present invention.

In another aspect the present invention relates to a method for improving the ability of a CAR to mediate serial killing of target cells when expressed in a T cell, which method comprises the step of altering the antigen-binding domain of the CAR such that the antigen-binding domain binds to its target antigen with an affinity in the range of 50 nM to 200 nM, wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.01 s⁻¹.

The altered antigen-binding domain may be an antigen-binding domain as defined the first aspect of the present invention.

The affinity of the antigen-binding domain may be altered by mutagenesis, followed by in vitro selection for variants having the required affinity.

In another aspect the present invention relates to an altered antigen-binding domain which has a modified affinity for its target antigen, wherein the modified affinity is in the range of 50 nM to 200 nM, and wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.01 s⁻¹.

A corresponding unaltered antigen-binding domain may have an affinity of greater than 200 nM, wherein said affinity comprises component kinetics such that the association rate constant (kₒₙ) is less than 1×10⁵ M⁻¹ s⁻¹, and/or the dissociation rate constant (k_{off}) less than 0.01 s⁻¹.

The altered antigen-binding domain is an antigen-binding domain as defined in the first aspect of the present invention.

In another aspect the present invention provides a method for treating cancer which comprises the step of administering a cell, a cell composition or a pharmaceutical composition according to the present invention to a subject.

The method may comprise the step of transducing or transfecting cells from the subject ex vivo with a vector according to the invention, then administering transfected cells back to the subject.

In another aspect the present invention provides a pharmaceutical composition according to the present invention for use in treating cancer.

In a further aspect the present invention relates to the use of a cell according to the invention in the manufacture of a pharmaceutical composition for treating cancer.

### DESCRIPTION OF THE FIGURES

FIG. 1—Diagram of Chimeric Antigen Receptors. Chimeric Antigen Receptors typically contain an ectodomain, a transmembrane domain and an endodomain. The ectodomain is formed by fusing an antigen-binding domain (typically an scFv) to a spacer domain. The endodomain can contain one or more signalling domains.

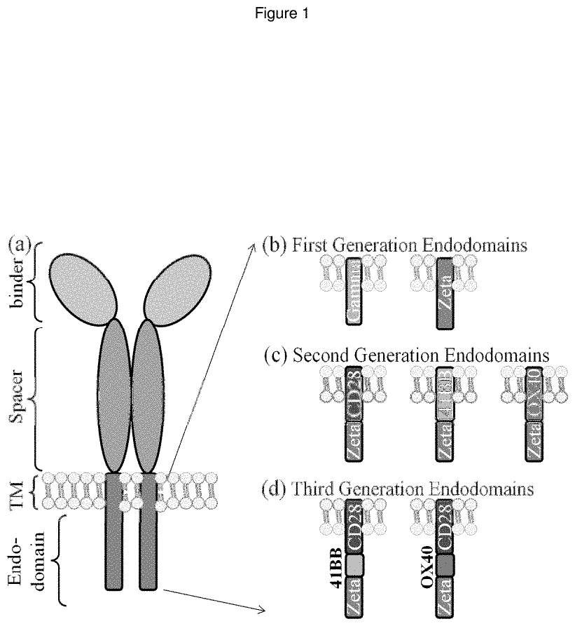

FIG. 2—Binding kinetics of different CD19 scFvs. Several CD19 specific scFvs including 4G7, fmc63 and CAT19 were tested for binding to recombinant CD19 using biocore. The data are plotted in a composite plot with on rate on the y-axis, off rate on the x-axis and the diagonal lines showing the Kd.

FIGS. 3A-3B—Stability of different CD19 scFvs as CARs. Primary human T-cells were transduced with CARs based on fmc63, 4G7 and CAT19 scFvs in the Campana format (Imai et al.; Leuk. Off. J. Leuk. Soc. Am. Leuk. Res. Fund UK 18, 676-684 (2004). T-cells were stained with anti-CD3 antibodies and for CAR with recombinant CD19. T-cells were analysed using flow-cytometry. Scatter plots (top) of CD3 vs CAR and histogrammes of just CAR expression are shown for CAR T-cells and non-transduced controls. Equal mean fluorescent intensity of the different CAR T-cells indicate equal stability of the different CD19 scFvs.

FIGS. 4A-4C—Chromium release assay. Primary human T-cells expressing CARs derived from fmc63, 4G7 or CAT19 were challenged with three different targets: (FIG. 4a) SupT1 cells which normally do not express CD19; (FIG. 4b) SupT1.CD19-SupT1 cells which have been engineered to express CD19 and (FIG. 4c) Raji cells which are a B-cell lymphoma line which normally express CD19. Targets were loaded with ⁵¹Cr and incubated at different effector:target ratios with CAR T-cells or non-transduced T-cells as controls for 4 hours. Supernatant was harvested and counted in a gamma counter and cytolysis determined.

FIG. 5—Proliferation and Cytokine release of CAR T-cells. CAR T-cells were co-cultured 1:1 with irradiated Raji cells after labelling of CAR T-cells with tritiated thymidylate. After 4 days, cells were pelleted and washed and tritium counting performed and proliferation determined by thymidylate incorporation. In an additional experiment T-cells and target cells were incubated 1:1 and at 72 hours, supernatant was assayed for a range of cytokines by cytokine bead array

FIG. 6—CD19 density in different cell lines. Using fluorescent beads with known fluorescent intensity, the CD19 surface copy number from a range of different cell lines was determined. A SupT1 CD19 variant termed SupT1 CD19 low had very low levels of CD19 at approximately 140 copies per cell.

FIG. 7—Flow based killing assays at low E:T. CAR T-cells and target cells were co-cultured for 24 hours at either one T-cell per target cell or one T-cell per 10 target cells. Cell kill was determined by flow-cytometry. Remaining live target cells is shown.

FIG. 8—Video microscopy. fmc64 and CAT19 T-cells were fluorescently labelled and incubated with Raji cells. Serial images were taken and cell motility across each frame measured. The duration of each tracked movement as well as its magnitude is plotted. Numeric data is also presented.

FIG. 9—In vivo model. Nalm6 cells were engrafted in NSG mice and CAR T-cells administered using conditions determined to be challenging for the CAR T-cells (i.e. only half of the mice expected to have complete responses). At 14 days after CAR T-cell administration, CAR T-cell numbers in bone-marrow (site of disease) were quantified. Each dot represents one mouse.

### DETAILED DESCRIPTION

Chimeric Antigen Receptors

Chimeric antigen receptors (CARs), also known as chimeric T cell receptors, artificial T cell receptors and chimeric immunoreceptors, are engineered receptors, which graft an arbitrary specificity onto an immune effector cell. In a classical CAR, the specificity of a monoclonal antibody is grafted on to a T cell. CAR-encoding nucleic acids may be transferred to T cells using, for example, retroviral vectors. In this way, a large number of cancer-specific T cells can be generated for adoptive cell transfer. Phase I clinical studies of this approach show efficacy.

The target-antigen binding domain of a CAR is commonly fused via a spacer and transmembrane domain to an endodomain. The endodomain may comprise or associate with an intracellular T-cell signalling domain. When the CAR binds the target-antigen, this results in the transmission of an activating signal to the T-cell it is expressed on. The CAR may also comprise an extracellular hinge and spacer element.

Binding Kinetics

The antigen binding domain is the portion of the CAR which recognizes antigen.

Binding affinity may be defined as the strength of binding of a single molecule to its target ligand. It is typically measured and reported by the equilibrium dissociation constant (K_{D}), which is used to evaluate and rank order strengths of bimolecular interactions. The binding of an antibody (or similar molecule)—to its antigen is a reversible process, and the rate of the binding reaction is proportional to the concentrations of the reactants. At equilibrium, the rate of [antibody] [antigen] complex formation is equal to the rate of dissociation into its components [antibody]+[antigen]. The measurement of the reaction rate constants can be used to define an equilibrium or affinity constant (1/K_{D}). The smaller the K_{D} value the greater the affinity of the antibody for its target.

As used herein, the terms “binding affinity” and “affinity” may be synonymous.

The Dissociation constant of antibody (K_{D}) is the ratio of the antibody dissociation rate (k_{off} or off-rate), how quickly it dissociates from its antigen, to the antibody association rate (kₒₙ or on-rate) of the antibody, how quickly it binds to its antigen (see Kastritis et al.; J. R. Soc. Interface R. Soc; 2013; 10; 20120835).

Thus binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation, e.g. by the SPR method (Biacore). The rate constants corresponding to the association and the dissociation of a monovalent complex are referred to as the association rate constants kₐ (or kₒₙ) and dissociation rate constant k_{d}. (or k_{off}), respectively. K_{D} is related to kₐ and k_{d} through the equation K_{D}=k_{d}/kₐ.

Following the above definition binding affinities associated with different molecular interactions, e.g. comparison of the binding affinity of different antibodies for a given antigen, may be compared by comparison of the K_{D} values for the individual binding domain/antigen complexes.

Without wishing to be bound by theory, the present inventors consider that a CAR comprising an antigen-binding domain (also referred to herein as the binding region) with binding kinetics which enables it to quickly bind but quickly dissociates from its target antigen increases the activity of CAR cells through improved serial killing i.e. a CAR T-cell which moves rapidly killing one target after another and hence has increased clinical activity.

A CAR comprising an antigen-binding domain according to the present invention may facilitate improved serial killing of target cells when expressed in a T cell, for example.

Serial killing relates to the ability of a CAR cell (e.g. a CAR T cell) to migrate between and kill separate target cells expressing the antigen recognized by the CAR.

Improved serial killing may be determined by killing assays at very low effector:target ratios and/or by video microscopy (as shown in the present Examples). Suitable killing assays are well known in the art and include, for example, chromium release assays or flow-cytometry assays of cell mediated cytotoxicity (as described in present Example 2, for example). Suitable flow-cytometry compatible dyes which specifically stain live cells and can be used to determine cell mediated cytotoxicity are well known in the art and include, for example, propidium iodide.

For example, improved serial killing may mean that a CAR cell is capable of killing at least 2-fold, 5-fold, or 10-fold more target cells at low effector:target ratios.

The improved serial killing may be improved compared to a CAR comprising an antigen binding domain which is not embodied by the present invention. For example the serial killing may be improved compared to a corresponding CAR which targets the same antigen but which has an antigen binding domain which has an affinity of greater than 200 nM, wherein said affinity comprises component kinetics such that the association rate constant (kₒₙ) is less than 1×10⁵ M⁻¹ s⁻¹, and/or the dissociation rate constant (k_{off}) less than 0.01 s⁻¹.

A low effector:target ratio may refer to an effector:target ratio of 16:1, 8:1, 4:1 or 2:1.

A cell expressing a CAR comprising an antigen-binding domain as defined herein may kill at least 2-fold more target cells at an effector:target ratio of 16:1, 8:1, 4:1 or 2:1.

A cell expressing a CAR comprising an antigen-binding domain as defined herein may kill at least 5-fold more target cells at an effector:target ratio of 16:1, 8:1, 4:1 or 2:1.

A cell expressing a CAR comprising an antigen-binding domain as defined herein may kill at least 10-fold more target cells at an effector:target ratio of 16:1, 8:1, 4:1 or 2:1.

The target cell killing may be determined by a chromium release assay.

The target cell killing may be determined by a flow-cytometry based assay of cell mediated cytotoxicity.

The value of the dissociation constant can be determined directly by known methods, and can be computed even for complex mixtures by methods such as those, for example, set forth in Caceci et al. (Byte 9:340-362, 1984). For example, the K_{D} may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics and binding affinity of the antigen binding domain also can be assessed by standard assays known in the art, such as Surface Plasmon Resonance (SPR), e.g. by using a Biacore™ system.

A competitive binding assay can be conducted in which the binding of the antigen binding domain to the target is compared to the binding of the target by another ligand of that target, such as an antibody. The concentration at which 50% inhibition occurs is known as the Kᵢ. Under ideal conditions, the Kᵢ is equivalent to K_{D}. The Kᵢ value will never be less than the K_{D}, so measurement of Kᵢ can conveniently be substituted to provide an upper limit for K_{D}.

The present antigen-binding domain has an affinity in the range of 50 nM to 500 nM wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.01 s⁻¹.

The present antigen binding domain has an affinity in the range of 50 nM to 500 nM, for example the affinity may be 50 nM to 400 nM, 50 nM to 300 nM, 50 nM to 250 nM, 50 nM to 200 nM, 50 nM to 150 nM. 75 nM to 125 nM, 80 nM to 120 nM, 90 nM to 110 nM or 95 nM to 105 nM.

In one embodiment, the affinity may be about 100 nM.

The present antigen binding domain may have an association rate constant (kₒₙ) which is greater than or equal to 1×10⁵ M⁻¹ s⁻¹, for example the kₒₙ may be from 1×10⁵ M⁻¹ s⁻¹ to 1×10⁷ M⁻¹ s⁻¹. For example, the antigen binding domain may have an association constant (kₒₙ) from 1×10⁵ M⁻¹ s⁻¹ to 1×10⁷ M⁻¹ s⁻¹, 1×10⁵ M⁻¹ s⁻¹ to 5×10⁶ M⁻¹ s⁻¹, 1×10⁵ M⁻¹ s⁻¹ to 1×10⁶ M⁻¹ s⁻¹, 5×10⁵ M⁻¹ s⁻¹ to 1×10⁶ M⁻¹ s⁻¹.

In one embodiment, the association constant (kₒₙ) may be about 5×10⁶ M⁻¹ s⁻¹.

The present antigen binding domain may have a dissociation rate constant (k_{off}) which is greater than or equal to 0.01 s⁻¹, for example the k_{off} may be from 0.01 s⁻¹ to 0.50 s⁻, for example from 0.01 s⁻¹ to 0.40 s⁻¹, 0.01 s⁻¹ to 0.30 s⁻¹, 0.01 s⁻¹ to 0.20 s⁻¹, 0.01 s⁻¹ to 0.10 s⁻¹, or 0.05 s⁻¹ to 0.10 s⁻¹.

In one embodiment, the dissociation rate constant (k_{off}) is about 0.07 s⁻¹.

The one embodiment, the present CAR comprises an antigen-binding domain with an affinity in the range of 50 nM to 200 nM, wherein said affinity comprises component kinetics such that the association rate constant (kₒₙ) is greater than or equal to 5×10⁵ M⁻¹ s⁻¹, and/or the dissociation rate constant (k_{off}) is greater than or equal to 0.05 s⁻¹. In one embodiment, the association rate constant (kₒₙ) is about 6×10⁵ M⁻¹ s⁻¹, and/or the dissociation rate constant (k_{off}) is about 0.07 s⁻¹.

In one embodiment, the affinity is about 100 nM, wherein the association rate constant (kₒₙ) is about 6×10⁵ M⁻¹ s⁻¹, and/or the dissociation rate constant (k_{off}) is about 0.07 s⁻¹.

Antigen Binding Domain

The antigen-binding domain may be based on the antigen binding site of an antibody or an antibody mimetic. For example, the antigen-binding domain may comprise: a single-chain variable fragment (scFv) derived from a monoclonal antibody; a natural ligand of the target antigen; a peptide with sufficient affinity for the target; a single domain antibody; or an artificial single binder such as a Darpin (designed ankyrin repeat protein).

The antigen binding domain may comprise a domain which is not based on the antigen binding site of an antibody. For example the antigen binding domain may comprise an extracellular domain of a membrane anchored ligand or a receptor for which the binding pair counterpart is expressed on the tumour cell.

The antigen binding domain may be based on a natural ligand of the antigen.

The antigen binding domain may comprise an affinity peptide from a combinatorial library or a de novo designed affinity protein/peptide.

The binding domain may comprise or consist of the antigen binding site antibody, for example the binding domain may comprise or consist of scFv.

A scFv commonly comprises the light (VL) and heavy (VH) variable regions of an antibody joined by a flexible linker.

The scFv may be in the orientation VH-VL, i.e. the VH is at the amino-terminus of the CAR molecule and the VL domain is linked to the spacer and, in turn the transmembrane domain and endodomain.

ScFvs against tumour associated antigens (TAAs) have been used to produce CARs to redirect T cells against TAAs expressed at the surface of tumour cells from various malignancies including leukaemia, lymphomas and solid tumours. A major advantage of endowing T cells with non-MHC-restricted, antibody-derived specificity is that the potential target structures are no longer restricted to protein-derived peptides, but rather comprise every surface molecule on tumour cells including proteins with varying glycosylation patterns and non-protein structures such as gangliosides or carbohydrate antigens. Thus, the panel of potential tumour-specific targets is enlarged.

In one embodiment, the antigen binding domain may be based on a mouse anti-CD19 monoclonal antibody.

For example, the antigen binding domain may comprise:
a) a heavy chain variable region (VH) having complementarity determining regions (CDRs) with the following sequences:

(SEQ ID No. 1)
CDR1-GYAFSSS;
(SEQ ID No. 2)
CDR2-YPGDED
(SEQ ID No. 3)
CDR3-SLLYGDYLDY;

b) a light chain variable region (VL) having CDRs with the following sequences:

(SEQ ID No. 4)
CDR1-SASSSVSYMH;
(SEQ ID No. 5)
CDR2-DTSKLAS
(SEQ ID No. 6)
CDR3-QQWNINPLT.

It may be possible to introduce one or more mutations (substitutions, additions or deletions) into each CDR without negatively affecting CD19-binding activity. Each CDR may, for example, have one, two or three amino acid mutations.

The CDRs may be in the format of a single-chain variable fragment (scFv), which is a fusion protein of the heavy variable region (VH) and light chain variable region (VL) of an antibody, connected with a short linker peptide of ten to about 25 amino acids.

The CDRs may be grafted on to the framework of a human antibody or scFv. For example, the antigen binding domain may comprise a CD19-binding domain consisting or comprising one of the following sequences.

The CAR of the present invention may comprise the following VH sequence:

VH sequence from murine monoclonal antibody
SEQ ID No. 7
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSS

The CAR of the present invention may comprise the following VL sequence:

VL sequence from murine monoclonal antibody
SEQ ID No 8
QIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFG
AGTKLELKR

The CAR of the invention may comprise the following scFv sequence:

VH-VL scFv sequence from murine monoclonal
antibody
SEQ ID No 9
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSSGGGGSGGGGSGGGGSQIVLTQSPAIMSA
SPGEKVTMTCSASSSVSYMHWYQQKSGTSPKRWIYDTSKLASGVPDRFS
GSGSGTSYFLTINNMEAEDAATYYCQQWNINPLTFGAGTKLELKR

The present CAR may consist of or comprise one of the following sequences:

CAT19 CAR using “Campana” architecture (see Examples)
SEQ ID No. 10
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
CAT19 CAR with an OX40-Zeta endodomain
SEQ ID No. 11
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPTTTPAPRPPTPAPTIASQPLSLRPEACRPAAGGAVHTRGLDFACDIYIWA
PLAGTCGVLLLSLVITLYCRRDQRLPPDAHKPPGGGSFRTPIQEEQADAHSTLAKIRVKFSRS
ADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
CAT19 CAR with a OD28-Zeta endodomain
SEQ ID No. 12
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPTTTPAPRPPTPAPTIASQPLSLRPEACRPAAGGAVHTRGLDFACDIYIWA
PLAGTCGVLLLSLVITLYCRSKRSRLLHSDYMNMTPRRPGPTRKHYQPYAPPRDFAAYRSRVK
FSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
Third generation CD19 CAR
SEQ ID No. 13
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPTTTPAPRPPTPAPTIASQPLSLRPEACRPAAGGAVHTRGLDFACDIFWVL
VVVGGVLACYSLLVTVAFIIFWVRSKRSRLLHSDYMNMTPRRPGPTRKHYQPYAPPRDFAAYR
SRDQRLPPDAHKPPGGGSFRTPIQEEQADAHSTLAKIRVKFSRSADAPAYQQGQNQLYNELNL
GRREEYDVLDKRRGRDPEMGGKPRRKNPQEGLYNELQKDKMAEAYSEIGMKGERRRGKGHDGL
YQGLSTATKDTYDALHMQALPPR
CD19 CAR with IgG1 hinge spacer
SEQ ID No. 14
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPAEPKSPDKTHTCPPCPKDPKFWVLVVVGGVLACYSLLVTVAFIIFWVRSK
RSRLLHSDYMNMTPRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIGMKGERRRGKGHDG
LYQGLSTATKDTYDALHMQALPPR
CD19 CAR with hinge-CH2-CH3 of human IgG1 with FcR binding sites
mutated out
SEQ ID No. 15
MGTSLLCWMALCLLGADHADAQVQLQQSGPELVKPGASVKISCKASGYAFSSSWMNWVKQRPG
KGLEWIGRIYPGDEDTNYSGKFKDKATLTADKSSTTAYMQLSSLTSEDSAVYFCARSLLYGDY
LDYWGQGTTLTVSSGGGGSGGGGSGGGGSQIVLTQSPAIMSASPGEKVTMTCSASSSVSYMHW
YQQKSGTSPKRWIYDTSKLASGVPDRFSGSGSGTSYFLTINNMEAEDAATYYCQQWNINPLTF
GAGTKLELKRSDPAEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGKKDPKFWV
LVVVGGVLACYSLLVTVAFIIFWVRSKRSRLLHSDYMNMTPRRPGPTRKHYQPYAPPRDFAAY
RSRVKFSRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR

The present CAR may comprise a variant of the sequence shown as SEQ ID No. 7, 8, 9, 10, 11, 12, 13, 14 or 15 having at least 80, 85, 90, 95, 98 or 99% sequence identity, provided that the variant sequence retain the capacity to bind CD19 (when in conjunction with a complementary VL or VH domain, if appropriate).

Sequence identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed.

Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final sequence identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % sequence identity. The software typically does this as part of the sequence comparison and generates a numerical result.

The terms “variant” according to the present invention includes any substitution of, variation of, modification of, replacement of, deletion of or addition of one (or more) amino acids from or to the sequence providing the resultant amino acid sequence retains substantially the same activity as the unmodified sequence.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

A nucleic acid sequence or amino acid sequence as described herein may comprise, consist of or consist essentially of a nucleic acid sequence or amino acid sequence as shown herein.

Transmembrane Domain

The CAR of the invention may also comprise a transmembrane domain which spans the membrane. It may comprise a hydrophobic alpha helix. The transmembrane domain may be derived from CD28, which gives good receptor stability.

The transmembrane region of CARs may be derived from homo- or heterodimeric type I membrane proteins like CD4, CD8, CD28, CD3, or Fc gamma.

The transmembrane domain may comprise the sequence shown as SEQ ID No. 16.

SEQ ID No. 16
FWVLVVVGGVLACYSLLVTVAFIIFWV

Spacer

The CAR of the present invention may comprise a spacer sequence to connect the antigen-binding domain with the transmembrane domain and spatially separate the antigen-binding domain from the endodomain. A flexible spacer allows to the antigen-binding domain to orient in different directions to enable antigen binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

(hinge-CH2CH3 of human IgG1)
SEQ ID No. 17
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):
SEQ ID No. 18
ITTPAPRPPTPAPTIASQPLSLRPEACRPAAGGAVHIRGLDFACDI
(human IgG1 hinge):
SEQ ID No. 19
AEPKSPDKTHTCPPCPKDPK
(IgG1 Hinge-Fc)
SEQ ID No. 20
AEPKSPDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK
(IgG1 Hinge-Fc modified to remove Fc receptor
recognition motifs)
SEQ ID No. 21
AEPKSPDKTHTCPPCPAPPVA*GPSVFLFPPKPKIDTLMIARTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPGKKDPK

Modified residues are underlined; * denotes a deletion.

Intracellular T Cell Signaling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, endodomains from CD28, or OX40 or 41BB can be used with CD3-Zeta to transmit a proliferative/survival signal, or all three can be used together.

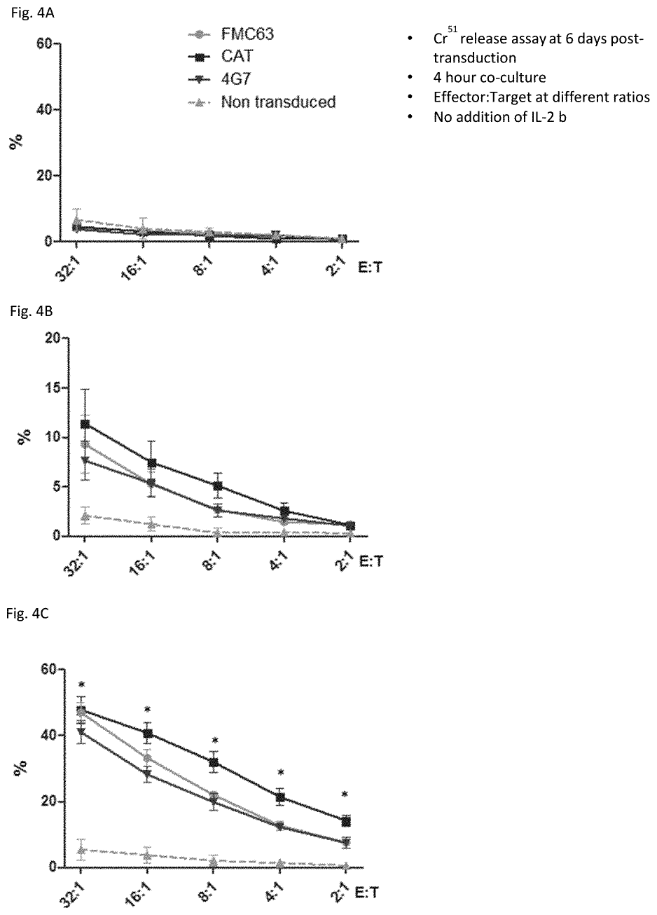

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains were constructed. Fusion of the intracellular part of a T-cell co-stimulatory molecule to that of CD3ζ resulted in second generation receptors which could transmit an activating and co-stimulatory signal simultaneously after antigen recognition. The co-stimulatory domain most commonly used was that of CD28. This supplies the most potent co-stimulatory signal, namely immunological signal 2, which triggers T-cell proliferation. Some receptors were also described which included TNF receptor family endodomains such as OX40 and 41BB which transmit survival signals. Finally, even more potent third generation CARs were described which had endodomains capable of transmitting activation, proliferation and survival signals. CARs and their different generations are summarized in FIG. 4.

The endodomain of the present CAR may be provided on a separate molecule to the antigen-binding domain, for example as described in the CAR signalling systems described in WO2015/150771, WO2016/030691 and WO2016/124930.

The endodomain of the present CAR may comprise combinations of one or more of the CD3-Zeta endodomain, the 41 BB endodomain, the OX40 endodomain or the CD28 endodomain.

The intracellular T-cell signalling domain (endodomain) of the CAR of the present invention may comprise the sequence shown as SEQ ID No. 22, 23, 24, 25, 26, 27, 28, or 29 or a variant thereof having at least 80% sequence identity.

(CD3 zeta endodomain)
SEQ ID No. 22
RSRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATK
DTYDALHMQALPPR
(41BB endodomain)
SEQ ID No. 23
KRGRKKLLYIFKQPFMRPVQTTQEEDGCSCRFPEEEEGGCEL
(OX40 endodomain)
SEQ ID No. 24
RRDQRLPPDAHKPPGGGSFRTPIQEEQADAHSTLAKI
(CD28 endodomain)
SEQ ID No. 25
KRSRLLHSDYMNMTPRRPGPTRKHYQPYAPPRDFAAY
Examples of combinations of such endodomains
include 41 BB-Z, OX40-Z, CD28-Z and
CD28-OX40-Zeta.
(41BB-Z endodomain fusion)
SEQ ID No. 26
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR
(OX40-Z endodomain fusion)
SEQ ID No. 27
RRDQRLPPDAHKPPGGGSFRTPIQEEQADAHSTLAKIRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
(CD28Z endodomain fusion)
SEQ ID No. 28
KRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
(CD28OXZ)
SEQ ID No. 29
KRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

A variant sequence may have at least 80%, 85%, 90%, 95%, 98% or 99% sequence identity to SEQ ID No. 22, 23, 24, 25, 26, 27, 28, or 29 provided that the sequence provides an effective transmembrane domain/intracellular T cell signaling domain.

Signal Peptide

The present CAR may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:

Signal peptide-antigen-binding domain-spacer domain-transmembrane domain/intracellular T cell signaling domain.

The signal peptide may comprise the SEQ ID No. 30 or a variant thereof having 5, 4, 3, 2 or 1 amino acid mutations (insertions, substitutions or additions) provided that the signal peptide still functions to cause cell surface expression of the CAR.

SEQ ID No. 30:
METDTLLLWVLLLWVPGSTG

The signal peptide of SEQ ID No. 30 is compact and highly efficient. It is predicted to give about 95% cleavage after the terminal glycine, giving efficient removal by signal peptidase.

Nucleic Acid

The present invention provides a nucleic acid sequence encoding a cell-surface antibody as described above. As used herein, the term nucleic acid sequence is synonymous with the term polynucleotide.

The nucleic acid sequence may be an RNA or DNA sequence or a variant thereof.

The nucleic acid sequence may encode a CAR according to the third aspect of the invention. In this respect, the nucleic acid sequence may comprise a sequence encoding an antibody domain operably linked to a sequence encoding a signalling domain.

The nucleic acid sequence may also comprise a nucleic acid sequence encoding a hinge region; a nucleic acid sequence encoding a spacer; and/or a nucleic acid sequence encoding a transmembrane region.

Where the nucleic acid sequence encodes a plurality of distinct sequences, such as VL and VH antibody domains, or cytoplasmic signalling domains; the nucleic acid sequence may comprise a plurality of separate sequences; a single sequence capable of producing more than one product (e.g. joined by an IRES); or a single sequence capable of producing a fused product (e.g. an scFv).

Vector

The present invention also provides a vector which comprises a nucleic acid sequence according to the present invention. Such a vector may be used to introduce the nucleic acid sequence into a host cell so that it expresses and produces a molecule according to the first aspect of the invention.

The vector may, for example, be a plasmid or a viral vector, such as a retroviral vector or a lentiviral vector.

The vector may be capable of transfecting or transducing a cell, such as a T cell.

Cell The invention also provides a cell which comprises a nucleic acid/polynucleotide according to the invention. The invention provides a cell which expresses a CAR according to the first aspect of the invention at the cell surface.

The cell may be a cytolytic immune cell, such as a T-cell or natural killer (NK) cell.

A cell capable of expressing a CAR according to the invention may be made by transducing or transfecting a cell with CAR-encoding nucleic acid.

The CAR-expressing cell of the invention may be generated ex vivo. The cell may be from a cell sample, such as a peripheral blood mononuclear cell (PBMC) sample from the patient or a donor. Cells may be activated and/or expanded prior to being transduced with CAR-encoding nucleic acid, for example by treatment with an anti-CD3 monoclonal antibody.

Pharmaceutical Composition

The present invention also relates to a pharmaceutical composition containing a CAR-expressing cell, or plurality of cells, of the invention together with a pharmaceutically acceptable carrier, diluent or excipient, and optionally one or more further pharmaceutically active polypeptides and/or compounds. Such a formulation may, for example, be in a form suitable for intravenous infusion.

Method of Treatment

CAR-expressing cells of the present invention may be capable of killing cancer cells, such as B-cell lymphoma cells. CAR-expressing cells, such as T-cells or NK cells, may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party). Alternatively, CAR-expressing cells may be derived from ex vivo differentiation of inducible progenitor cells or embryonic progenitor cells to cells such as T-cells. In these instances, CAR cells are generated by introducing DNA or RNA coding for the CAR by one of many means including transduction with a viral vector, transfection with DNA or RNA.

T or NK cells expressing a CAR molecule of the present invention may be used for the treatment of a cancerous disease, in particular a cancerous disease associated with CD19 expression.

A method for the treatment of disease relates to the therapeutic use of a cell or population of cells of the invention. In this respect, the cells may be administered to a subject having an existing disease or condition in order to lessen, reduce or improve at least one symptom associated with the disease and/or to slow down, reduce or block the progression of the disease. The method of the invention may cause or promote cell mediated killing of CD19-expressing cells, such as B cells.

Method

The present invention also provides a method for selecting an antigen-binding domain for use in a chimeric antigen receptor (CAR), the method comprising:
(a) determining the affinity and affinity component kinetics of the antigen-binding domain; and
(b) selecting the antigen-binding domain for use in a CAR if it has an affinity in the range of 50 nM to 200 nM, wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.1 s⁻¹.

The affinity and affinity component kinetics of the antigen-binding domain may be determined using the methods described herein, for example by Surface Plasmon Resonance (SPR), e.g. by using a Biacore™ system.

The method may comprise determining the affinity and affinity component kinetics of the antigen-binding domain of a plurality of antigen-binding domains.

A plurality of antigen-binding domains refers to two or more antigen-binding domains, for example, 2, 5, 10, 20 or more antigen-binding domains.

The antigen-binding domain selected may be an antigen-binding domain according to the present invention.

The present invention further provides a method for improving the ability of a CAR to mediate serial killing of target cells when expressed in a T cell, which method comprises the step of altering the antigen-binding domain of the CAR such that the antigen-binding domain binds to its target antigen with an affinity in the range of 50 nM to 200 nM, wherein said affinity comprises component kinetics such that the association rate constant (kₒₙ) is greater than or equal to 1×10⁵ M⁻¹ s⁻¹, and/or the dissociation rate constant (k_{off}) is greater than or equal to 0.01 s⁻¹.

In the present method, prior to alteration the antigen-binding domain may have an affinity of greater than 200 nM, wherein said affinity comprises component kinetics such that the association rate constant (kₒₙ) is less than 1×10⁵ M⁻¹ s⁻, and/or the dissociation rate constant (k_{off}) less than 0.01 s⁻¹.

The altered antigen-binding domain selected may be an antigen-binding domain according to the present invention.

Mutagenesis and Selection

Techniques for altering the affinity of antigen-binding domains (e.g. scFVs) are known in the art. For example, mutations may be introduced into the polynucleotide encoding the scFV, and the resulting variant antigen-binding domains screened for low-affinity binders, by a technique such as yeast display or phage display.

The mutation step may be random, or targeted to specific residues in the antigen binding pocket (e.g. via site-directed mutagenesis).

Suitable methods for generating altered antigen-binding domains include, but are not limited to

The process may involve successive rounds of mutagenesis and screening, for example as part of an in vitro evolution process.

Antigen

The term “antigen” in terms of target antigen means an entity which is recognised (i.e. binds specifically) to the antibody expressed at the T cell surface.

An “epitope” is the portion of a molecule which is recognised by antibody. In the sense of the present invention, an antigen is or comprises at least one epitope.

An antigen may be a complete molecule, or a fragment thereof. The antigen may be or be derivable from a naturally occurring molecule.

The antigen may be or be derivable from, for example, a protein, glycoprotein, glycolipid, or carbohydrate.

Where the CAR or a CAR expressing cell is for use in the treatment of cancer, the antigen-binding domain may recognise an antigen that is or is part of a tumour associated antigen (TAA).

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an agent” includes a plurality of such candidate agents and equivalents thereof known to those skilled in the art, and so forth.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Generation of CD19 CARs

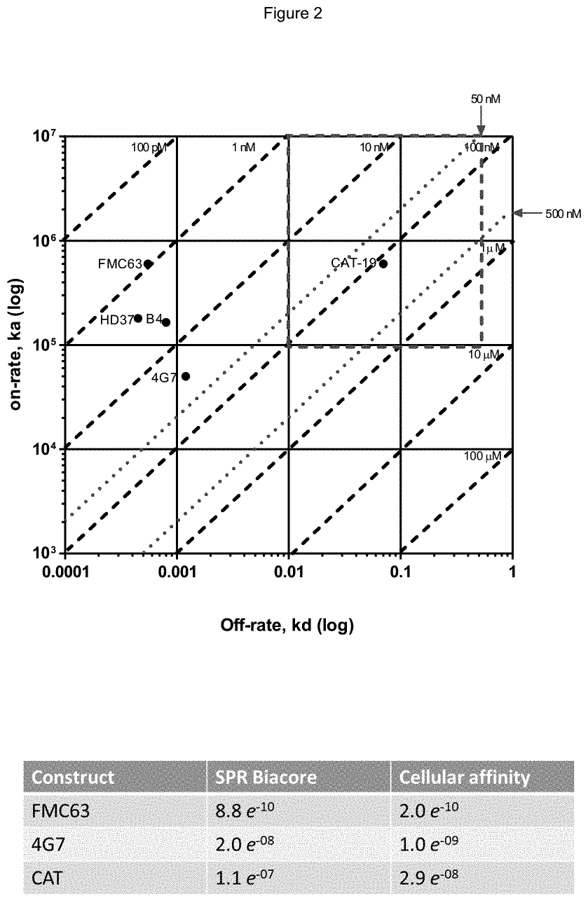

Three scFvs (Fmc63 (Imai et al.; as above), 4G7 (Poirot et al.; Cancer Res. 75, 3853-3864; 2015 and CAT19b) were generated as soluble recombinant proteins as scFv-Fc format in 293T cells. Recombinant soluble CD19 ectodomain was also generated from 293T cells. The binding kinetics of the three scFvs against CD19 was determined by biocore. The results are shown in FIG. 2.

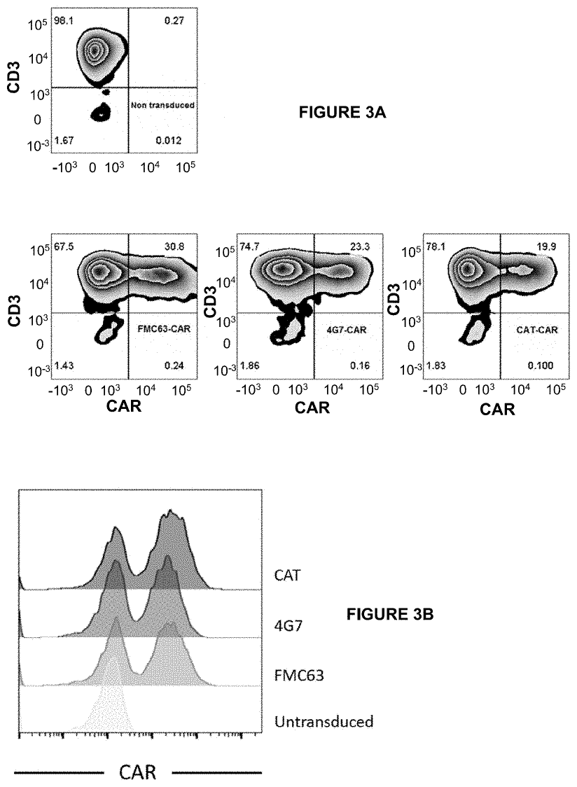

The three scFvs were expressed on a CAR format described by Campana (Imai et al.; as above) which contains a CD8 stalk as spacer and trans-membrane domain, the 41BB endodomain and the CD3-Zeta endodomain. T-cells were transduced with lentiviral vectors coding for these CARs and stained with recombinant CD19. The three scFvs were equally stable when expressed on the cell surface as a 41BB-Z CAR as determined by flow-cytometric analysis (FIG. 3).

### Example 2—Effector Functions of CAR T Cells Expressing Different CD19 CARs

Primary human T-cells were used in a classical cytotoxicity assay using either SupT1 cells (a T-cell line typically CD19 negative), SupT1 cells engineered to express CD19 and Raji cells (a B-cell lymphoma cell line which naturally expresses CD19. Target cells were loaded with ⁵¹Cr and washed. Non-transduced and CAR transduced T-cells were co-cultured with target cells for 4 hours with different effector to target ratios. Supernatant was harvested and gamma count thereof used to determine killing of target cells. Killing was identical across the different CD19 CARs (FIG. 4).

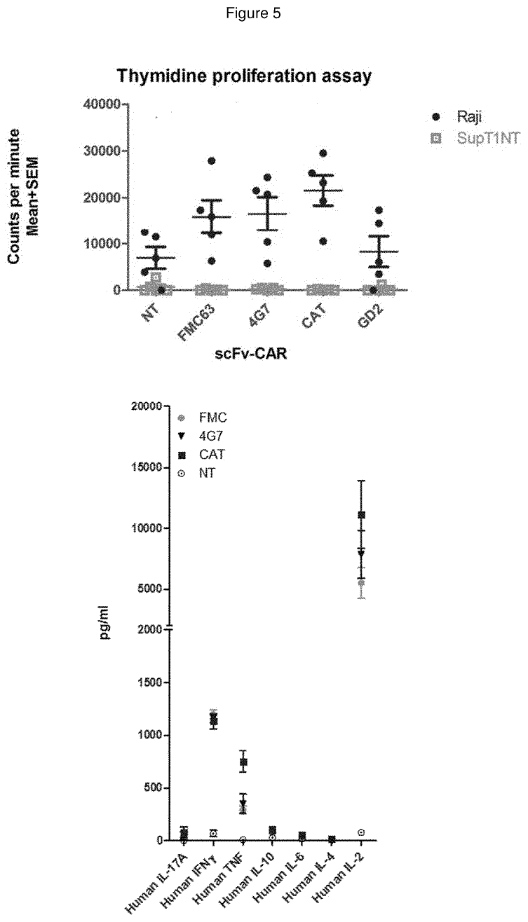

Next, proliferation was determined by measuring incorporation of tritiated thymidylate. CAR T-cell/irradiated target cells were co-cultured. Tritiated thymidylate was added to the co-culture. After 4 days, tritium content of cell lysate was determined by liquid scintillation counting which indicated incorporation of thymidylate and hence proliferation. This showed that CAT19 CAR T-cells proliferated more than fmc63 or 4G7 CAR T-cells. Cytokine release after CD19+ target cell encounter was also measured using a cytokine bead array. CAT19 CAR T-cells secreted more TNF and IL2 than the two other types of CD19 CAR T-cells (FIG. 5).

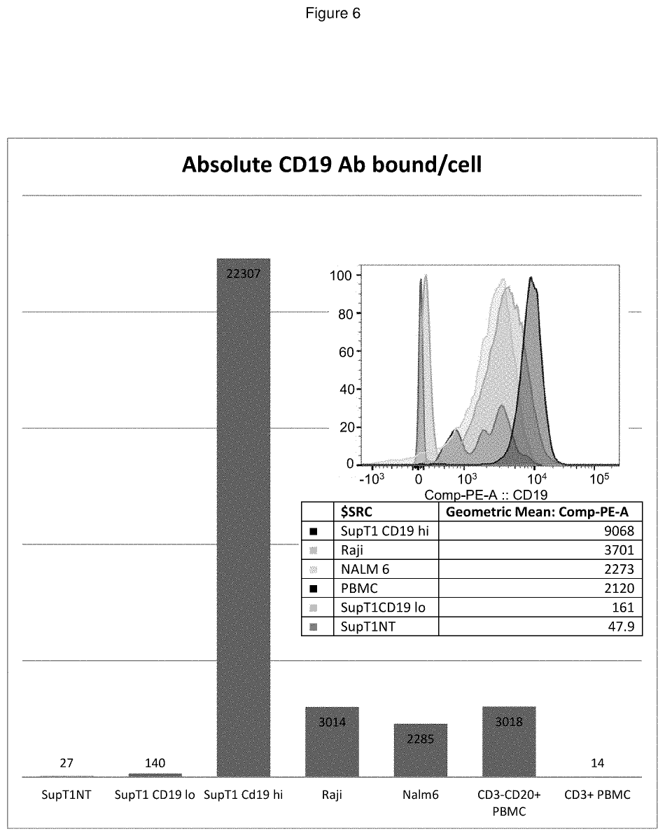

CD19 expression density may influence function of CAR T-cells. A SupT1 cell clone was established which expressed very low levels of CD19. The copy number of CD19 on various target cell including the previously used SupT1.CD19 and the very low CD19 expressing SupT1 cells was determined by correlating fluorescence measurements by flow cytometery from CD19 stained cells against the fluorescence from control quantification beads. NALM6 and SupT1 CD19 low cells were selected for further study since their low CD19 density should make CAR recognition and triggering particularly challenging (FIG. 6).

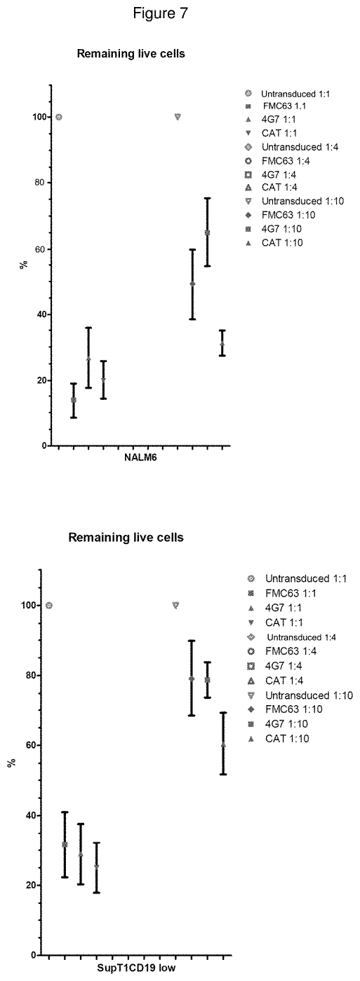

Low effector:target assays were next performed. These were designed to be as challenging to CAR function as possible and measure the ability of CAR T-cells to repeatedly kill (serial killing). Transduced T-cells were incubated with target cells (either NALM6 or SupT1 CD19 low) at reducing effector to target ratio. The most challenging ratio was one T-cells for every 10 target cells. Twenty-four hours after co-incubation, the cultures were studied by flow-cytometry and the numbers of target cells left alive determined. At standard E:T ratio of 1:1, all CARs killed equally effectively suggesting that the CAT19 does not particularly confer an advantage at killing low-density target cells. However, CAT19 T-cells effected considerable cell kill at very low E:T ratios with superior killing to fmc63 and 4G7 CAR T-cells (FIG. 7).

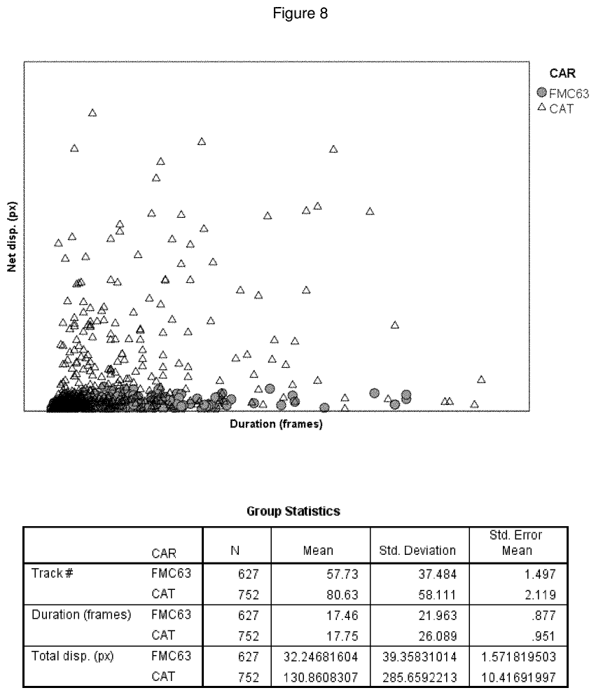

Next, video microscopy of these co-cultures were undertaken. CAR T-cells were fluorescently labelled and their behaviour during co-culture with Raji target cells determined. CAT19 CAR T-cells had considerably higher motility than fmc63 CAR T-cells (FIG. 8).

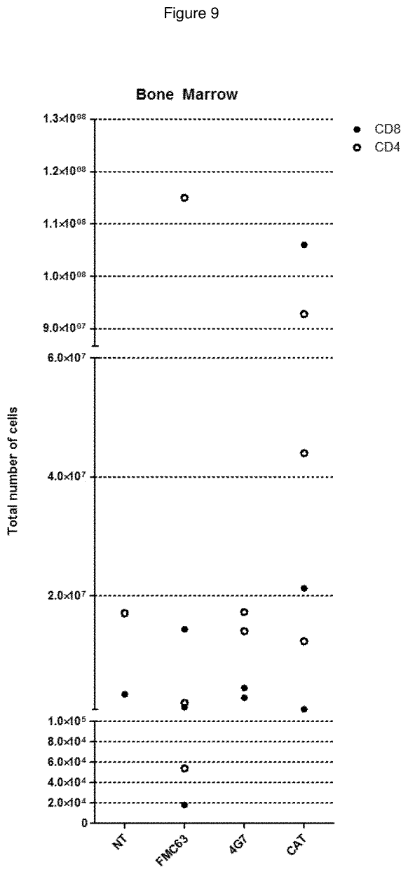

Finally, a mouse model of leukaemia was established which like the low E:T ratio challenged CAR T-cell activity. We determined the extent of NALM6 xenograft burden/T-cell dose in NOD.Cg-Prkdc^{scid} Il2rg^{tm1Wjl}/SzJ (NSG) mice where only half the mice would experience elimination of the xenograft after CAR T-cell therapy. Under these conditions, CAT19 T-cells were more abundant in the bone-marrow of mice after eradication of the malignant cells (FIG. 9).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments.

Indeed, various modifications of the described modes for carrying out the invention which are obvious to those skilled in cancer therapy, immunology, molecular biology or related fields are intended to be within the scope of the following claims.

1-28. (canceled)

29. A method of treating a cancer which comprises the step of administering a cell which comprises a chimeric antigen receptor (CAR) to a subject, wherein the CAR comprises an antigen-binding domain with an affinity in the range of 50 nM to 500 nM, wherein said affinity comprises component kinetics such that the association rate constant (kₒₙ) is greater than or equal to 1×10⁵ M⁻¹ s⁻¹, and the dissociation rate constant (k_{off}) is greater than or equal to 0.01 s⁻¹.

30. The method according to claim 29, wherein the affinity comprises component kinetics such that the association rate constant (kₒₙ) is from 1×10⁵ M⁻¹ s⁻¹ to 1×10⁷ M⁻¹ s⁻¹, and the dissociation rate constant (k_{off}) is from 0.01 s⁻¹ to 0.5 s⁻¹.

31. The method according to claim 29, wherein the antigen-binding domain is a scFv.

32. The method according to claim 29, wherein the antigen is CD19.

33. The method according to claim 29, wherein the cancer is associated with CD19 expression

34. The method according to claim 29, wherein the cancer associated with CD19 expression is a B cell lymphoma or leukemia.

35. The method according to claim 29, wherein the cell is a T cell or a natural killer (NK) cell.

36. The method according to claim 29, which comprises the following steps:
i) transducing or transfecting cells from the subject ex vivo with a vector which comprises a polynucleotide which encodes a CAR as defined in claim 29, and
ii) administering transfected cells back to the subject.

37. A method for selecting an antigen-binding domain for use in a chimeric antigen receptor (CAR), the method comprising:
a) determining the affinity and affinity component kinetics of the antigen-binding domain; and
b) selecting the antigen-binding domain for use in a CAR if it has an affinity in the range of 50 nM to 200 nM,
wherein said affinity comprises component kinetics such that the association rate constant (kₒₙ) is greater than or equal to 1×10⁵ M⁻¹ s⁻¹, and the dissociation rate constant (k_{off}) is greater than or equal to 0.01 s⁻¹.

38. The method according to claim 37, wherein the affinity comprises component kinetics such that the association rate constant (kₒₙ) is from 1×10⁵ M⁻¹ s⁻¹ to 1×10⁷ M⁻¹ s⁻¹, and the dissociation rate constant (k_{off}) is from 0.01 s⁻¹ to 0.5 s⁻¹.

39. The method according to claim 37, wherein the antigen-binding domain is a scFv.

40. The method according to claim 37, wherein the antigen is CD19.


SEQUENCE LISTING (30 sequences)

SEQ ID NO: 1 - protein, 7 aa, Artificial Sequence
  heavy chain variable region (VH) with CDR1
GYAFSSS

SEQ ID NO: 2 - protein, 6 aa, Artificial Sequence
  heavy chain variable region (VH) with CDR2
YPGDED

SEQ ID NO: 3 - protein, 10 aa, Artificial Sequence
  heavy chain variable region (VH) with CDR3
SLLYGDYLDY

SEQ ID NO: 4 - protein, 10 aa, Artificial Sequence
  light chain variable region (VL) with CDR1
SASSSVSYMH

SEQ ID NO: 5 - protein, 7 aa, Artificial Sequence
  light chain variable region (VL) with CDR1
DTSKLAS

SEQ ID NO: 6 - protein, 9 aa, Artificial Sequence
  light chain variable region (VL) with CDR1
QQWNINPLT

SEQ ID NO: 7 - protein, 119 aa, Artificial Sequence
  VH sequence from murine monoclonal antibody
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 8 - protein, 107 aa, Artificial Sequence
  VL sequence from murine monoclonal antibody
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 9 - protein, 241 aa, Artificial Sequence
  VH-VL scFv sequence from murine monoclonal antibody
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 10 - protein, 488 aa, Artificial Sequence
  CAT19 CAR using “Campana” architecture
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 483 aa, Artificial Sequence
  CAT19 CAR with an OX40-Zeta endodomain
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 487 aa, Artificial Sequence
  CAT19 CAR with a CD28-Zeta endodomain
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 527 aa, Artificial Sequence
  Third generation CD19 CAR
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 465 aa, Artificial Sequence
  CD19 CAR with IgG1 hinge spacer
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 681 aa, Artificial Sequence
  CD19 CAR with hinge-CH2-CH3 of human IgG1 with FcR binding sites mutated out
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 27 aa, Artificial Sequence
  transmembrane domain
FWVLVVVGGVLACYSLLVTVAFIIFWV

SEQ ID NO: 17 - protein, 234 aa, Artificial Sequence
  hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 18 - protein, 46 aa, Artificial Sequence
  human CD8 stalk
TTTPAPRPPTPAPTIASQPLSLRPEACRPAAGGAVHTRGLDFACDI

SEQ ID NO: 19 - protein, 20 aa, Artificial Sequence
  human IgG1 hinge
AEPKSPDKTHTCPPCPKDPK

SEQ ID NO: 20 - protein, 237 aa, Artificial Sequence
  IgG1 Hinge-Fc
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 21 - protein, 236 aa, Artificial Sequence
  IgG1 Hinge - Fc modified to remove Fc receptor recognition motifs
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 22 - protein, 114 aa, Artificial Sequence
  CD3 zeta endodomain
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 42 aa, Artificial Sequence
  41BB endodomain
KRGRKKLLYIFKQPFMRPVQTTQEEDGCSCRFPEEEEGGCEL

SEQ ID NO: 24 - protein, 37 aa, Artificial Sequence
  OX40 endodomain
RRDQRLPPDAHKPPGGGSFRTPIQEEQADAHSTLAKI

SEQ ID NO: 25 - protein, 37 aa, Artificial Sequence
  CD28 endodomain
KRSRLLHSDYMNMTPRRPGPTRKHYQPYAPPRDFAAY

SEQ ID NO: 26 - protein, 154 aa, Artificial Sequence
  41BB-Z endodomain fusion
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 149 aa, Artificial Sequence
  OX40-Z endodomain fusion
RRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 151 aa, Artificial Sequence
  CD28Z endodomain fusion
KRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 187 aa, Artificial Sequence
  CD28OXZ
KRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 20 aa, Artificial Sequence
  signal peptide
METDTLLLWVLLLWVPGSTG